(19) United States
(12) Patent Application Publication
(10) Pub. No.: US 2026/0048085 A1
(43) Pub. Date: Feb. 19, 2026

(54) MESENCHYMAL STEM CELLS FOR USE IN THE TREATMENT OF INSECT-BITE HYPERSENSITIVITY IN EQUINES

(71) Applicant: BOEHRINGER INGELHEIM VETMEDICA GMBH, Ingelheim am Rhein (DE)

(72) Inventors: Holger RICHLY, Ingelheim am Rhein (DE); Elisabeth RUZIN, Ingelheim am Rhein (DE); Eleonora MAZZOTTI, Ingelheim am Rhein (DE)

(21) Appl. No.: 19/101,796

(22) PCT Filed: Aug. 10, 2023
(86) PCT No.: PCT/EP2023/072139

§ 371 Date: Feb. 6, 2025

(30) Foreign Application Priority Data
Aug. 11, 2022 (EP) 22190046.7

(51) Int. Cl. A61K 35/28; A61P 37/08

(52) U.S. Cl. CPC A61K 35/28; A61P 37/08

(57) ABSTRACT

The current invention relates to mesenchymal stem cells (MSCs) or a pharmaceutical composition comprising an effective amount of MSCs for use in the treatment of insect-bite hypersensitivity (IBH) in equines.

### FIELD OF THE INVENTION

The present invention relates to mesenchymal stem cells for use in the treatment of insect-bite hypersensitivity (IBH) in equines.

### BACKGROUND

Insect bite hypersensitivity (IBH) is the most common allergic skin disease of horses and ordinarily manifests as a chronic relapsing seasonal allergic dermatitis. IBH is initially presented as pruritic dermatitis frequently affecting the head, mane, belly (including chest), back or tail area followed by self-trauma leading to hair loss and excoriations, which contribute to development of secondary bacterial infections. It is caused by bites of insects mostly of the midge genus Culicoides. IBH has been described worldwide, except in Iceland and New Zealand, and affects approximately 10% of horses of all breeds. Affected horses often develop, specifically in summer, severe skin lesions due to self-mutilation in an attempt to alleviate the itch. Severely affected horses are not suitable for riding or showing purposes because of the extreme discomfort and disfigurement. This can also happen to less affected horses depending on the location of the lesions. The commercial value of those horses is also significantly reduced and the disease is a significant animal welfare issue in severely affected equids. Horses with severe symptoms are often euthanized.

Although IBH is the most common allergic skin disease of horses, the only efficient treatment available is, beside reduction of exposure to Culicoides by stabling, use of blankets or repellents, symptomatic therapy using glucocorticoids. Corticosteroids, temporarily inhibit inflammation mediators and immune cells, however, long-term treatment causes various severe adverse effects. Too high allergic pressure and intolerance also limit the application in some patients.

Topical treatments can sometimes alleviate clinical signs of IBH. A cream containing omega-3-fatty acids, humectants, and emollients has been shown to improve clinical lesions of IBH by providing missing elasticity to the dried skin but did not influence the pruritic score and continuation of the disease. Furthermore, antihistamines and allergen-specific immunotherapy (ASIT) seem to be of limited efficacy in the treatment of IBH.

Although IBH was first described in 1840 and is currently the best characterized allergic disease in horses, treatment options are still poor, and currently, no satisfactory and safe treatment of IBH is available.

The present invention aims to provide a safe and efficacious treatment, thus resolving at least some of the problems and disadvantages mentioned above.

### SUMMARY OF THE INVENTION

The present invention and embodiments thereof serve to provide a solution to one or more of above-mentioned disadvantages. To this end, the present invention relates to mesenchymal stem cells (MSCs) or a pharmaceutical composition comprising an effective amount of MSCs for use in the treatment of insect-bite hypersensitivity (IBH) in equines according to claim 1. In embodiments, said MSCs are derived from blood, preferably peripheral blood. In embodiments, said MSCs are intravenously administered. In embodiments, said MSCs are native MSCs. In embodiments, said MSCs are allogeneic MSCs. Preferred embodiments of the MSCs for use of the invention are shown in any of the claims 2-16.

Currently no satisfactory treatment for IBH is available. The inventors surprisingly found that MSCs could be effectively used in the treatment of insect-bite hypersensitivity (IBH) in equines.

### DESCRIPTION OF FIGURES

FIG. 1 shows a schematic representation of the twelve regions of the equine that are scored for seven symptoms (wheals, papules, broken hairs, alopecia, crusts/scabing, blood/exudate and purulence) according to a scoring system according to and/or modified after Geiben used to evaluate IBH-associated clinical symptoms according to an embodiment of the present invention.

FIG. 2 shows data from example 1, depicting the clinical score for the entire group of horses (median and range, n=3) for the same period of time in 2021 (the horses did not receive the composition for use according to an embodiment of the present invention, negative control) and in 2022 (the horses received the composition for use according to an embodiment of the present invention). Grey enveloped circles refer to the treatment year 2022, and black circles refer to the untreated year 2021. Arrows depict the injection time points.

### DETAILED DESCRIPTION OF THE INVENTION

The present invention concerns mesenchymal stem cells (MSCs) or a pharmaceutical composition comprising an effective amount of MSCs for use in the treatment of insect-bite hypersensitivity (IBH) in equines.

Currently, no satisfactory treatment for IBH is available. The inventors surprisingly found that MSCs could be effectively used in the treatment of insect-bite hypersensitivity (IBH) in equines.

### Definitions

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or >7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The terms “mesenchymal stem cells” or “MSCs” refer to multipotent, self-renewing cells that express a specific set of surface antigens and can differentiate into various cell types, including but not limited to adipocytes, chondrocytes, and osteocytes when cultured in vitro or when present in vivo.

The term “isolated”, refers to both the physical identification and isolation of a cells from a cell culture or a biological sample, like blood, that can be performed by applying appropriate cell biology technologies that are either based on the inspection of cell cultures and on the characterization (and physical separation when possible and desired) of cells corresponding to the criteria, or on the automated sorting of cells according to the presence/absence of antigens and/or cell size (such as by FACS). In some embodiments, the terms “isolating” or “isolation” may comprise a further step of physical separation and/or quantification of the cells, especially by carrying out flow cytometry.

The term “in vitro” as used herein denotes outside, or external to, a body. The term “in vitro” as used herein should be understood to include “ex vivo”. The term “ex vivo” typically refers to tissues or cells removed from a body and maintained or propagated outside the body, e.g., in a culture vessel or a bioreactor.

The term “passage” or “passaging” is common in the art and refers to detaching and dissociating the cultured (mesenchymal stem) cells from the culture substrate and from each other. For sake of simplicity, the passage performed after the first time of growing the cells under adherent culture conditions is generally referred to as “first passage” (or passage 1, P1). The cells may be passaged at least one time and preferably two or more times. Each passage subsequent to passage 1 is referred to with a number increasing by 1, e.g., passage 2, 3, 4, 5, or P1, P2, P3, P4, P5, etc.

The term “cell medium” or “cell culture medium” or “medium” refers to an aqueous liquid or gelatinous substance comprising nutrients which can be used for maintenance or growth of cells. Cell culture media can contain serum or be serum-free. The cell medium may comprise or be supplemented with growth factors.

The term “growth factor” as used herein refers to a biologically active substance which influences proliferation, growth, differentiation, survival and/or migration of various cell types, and may affect developmental, morphological and functional changes in an organism, either alone or when modulated by other substances. A growth factor may typically act by binding, as a ligand, to a receptor (e.g., surface or intracellular receptor) present in cells.

“Autologous” administration of MSCs in the present context refers to MSCs from a donor being administered to a recipient, wherein both recipient and donor are the same.

“Allogeneic” administration of MSCs in the present context refers to MSCs from a donor being administered to a recipient, wherein both recipient and donor are of the same species, but are not the same.

“Native MSCs” in the context of the present invention refers to MSCs which have not first in vitro been exposed to a stimulating agent, such as inflammatory mediators, an inflammatory environment or one or more growth factors. Such inflammatory environment refers to a state or condition characterized by (i) an increase of at least one pro-inflammatory immune cell, pro-inflammatory cytokine, or pro-inflammatory chemokine; and (ii) a decrease of at least one anti-inflammatory immune cell, anti-inflammatory cytokine, or anti-inflammatory chemokine.

The term “anti-inflammatory”, “anti-inflammation”, “immunosuppressive”, and “immunosuppressant” refers to any state or condition characterized by a decrease of at least one indication of localized inflammation (such as, but not limited to, heat, pain, swelling, redness, pruritus and loss of function) and/or a change in systemic state characterized by (i) a decrease of at least one pro-inflammatory immune cell, pro-inflammatory cytokine, or pro-inflammatory chemokine; and (ii) an increase of at least one anti-inflammatory immune cell, anti-inflammatory cytokine, or anti-inflammatory chemokine.

The “population doubling time” or “PDT” of current invention is to be calculated by the formula: PDT=T×In(N_{f}/Nᵢ)/In(2), whereby T is the cell culture time (in days) to reach 80% confluency, N_{f} is the final number of cells after cell detachment and whereby Nᵢ is the initial number of cells at time point zero.

By the term “anti-coagulant”, it is meant a composition that can inhibit the coagulation of the blood. Examples of anticoagulants used in the present invention include EDTA or heparin.

The term “buffy coat” in this invention, is to be understood as the fraction of non-coagulated blood, preferably obtained by means of a density gradient centrifugation, whereby the fraction is enriched with white blood cells and platelets.

The term “blood-inter-phase” is to be understood as that fraction of the blood, preferably obtained by means of a density gradient, located between the bottom fraction, mainly consisting of erythrocytes and polymorphonuclear cells, and the upper fraction, mainly consisting of plasma. The blood-interphase is the source of blood mononuclear cells (BMCs) comprising monocytes, lymphocytes, and MSCs.

The term “suspension diameter” as used herein, is understood as the mean diameter of the cells, when being in suspension. Methods of measuring diameters are known in the art. Possible methods are flow cytometry, confocal microscopy, image cytometer, or other methods known in the art.

The term “treatment” as used herein refers to prophylactic, metaphylactic and/or therapeutic measures to reduce or prevent pathological conditions or disorders from developing or progressing.

The terms “patient”, “subject”, “animal”, or “mammal” are used interchangeably and refer to a mammalian subject to be treated. Preferably, the mammal is an equine, such as a horse.

The term “allergy” or “allergic” refers to a disease following a response by the immune system to an otherwise innocuous antigen. Allergy is one of a class of immune system responses that are termed hypersensitivity reactions. The term “allergen” or “allergens” refer(s) to (a) substance(s) that cause(s) an allergic reaction.

The term “hypersensitivity reactions” refers to harmful immune responses that produce tissue injury and may cause serious disease.

The term “antigen” or “antigens” refer(s) to (a) substance(s) capable of inducing a specific immune response against that substance(s).

An “IBH season” as used herein refers to a certain period in a certain calendar year wherein the causative insect(s) are/is present and elicit(s) symptoms in sensitised equines. In an embodiment, an average of multiple seasons can be calculated and used in the comparison. In an embodiment, a comparison for the entire treatment group or for each individual equine is made between two (preferably consecutive) IBH seasons, wherein during a first IBH season the equine(s) did not receive the MSCs or the composition for use according to the current invention and during a second IBH season the equine(s) did receive the MSCs or the composition for use according to the current invention. “First” and “second” do not refer to a chronological order, the “first IBH season” can thus refer to a season (or average of seasons) occurring before or after the second IBH season.

### DESCRIPTION

Insect bite hypersensitivity (IBH), also known as sweet itch, summer eczema, or Queensland itch, is an allergic reaction of equines to bites of insects (mostly Culicoides spp.) and is observed in many countries worldwide. Different types of progressing skin lesions are typical symptoms of this seasonal and refractory chronic disease. From spring over summer to autumn, the skin symptoms are first of acute nature and then evolve to chronic nature. On a cellular level, the skin lesions are characterized by massive eosinophil infiltration caused by an underlying allergic response. Management of IBH currently is focused on avoiding exposure of the horse to those allergens wherever possible, treating secondary infections, and managing the itching if allergen exposure is unavoidable. Itching is usually controlled using topically applied sprays, shampoos, or lotions containing anti-inflammatory medications such as hydrocortisone, triamcinolone, or fluocinolone acetonide. In severe cases, stronger anti-inflammatory drugs such as prednisolone or dexamethasone can be used.

Corticosteroids have a powerful anti-inflammatory and anti-pruritic activity. Their activity, however, varies tremendously. There is no consistency in the individual reaction, not only in relation to the corticosteroid used, but also for the same corticosteroid. The effect is reduced over time and the doses required are increased. Steroids and cortisones can further cause severe side effects and secondary issues such as liver and kidney problems and laminitis. Laminitis is a secondary condition brought on by corticosteroids and is a painful and potentially crippling disease that can be fatal to equines. The condition causes changes in the blood supply to the hoof and disruption to the interconnecting support tissues within the hoof; resulting in rotation of the pedal bone. Rotation of the pedal bone if severe enough is irreversible and can result in euthanasia.

Furthermore, antihistamines and allergen-specific immunotherapy (ASIT), seem to be of limited efficacy in the treatment of IBH. Antihistamines only address one of the inflammatory mediators of this complex disease. ASIT is known from the human field to have a variable and unpredictable outcome, depending on the patient.

Mesenchymal stem cells (MSCs) have been proposed for use in the treatment of inflammatory-related diseases because of their immunomodulatory properties.

These immunomodulatory properties could prevent or suppress the exaggerated inflammation process, slow down its progression on a very short term and even cause a reversion of the inflammation process.

Without wishing to be bound to theory, in contrast to other inflammatory-related diseases, IBH presents unique treatment difficulties. First, IBH is a wrongly directed and even exaggerated inflammation process to an otherwise completely harmless antigen, the allergen. Hence, from the start of IBH in its allergen-sensitisation phase, the processes are pathologic and not supporting self-defense or trauma healing. Secondly, IBH is considered to be a multifactorial disease with genetic and environmental factors contributing to onset and perpetuation of the disease. A third difficulty concerns the huge surface area of the patient which can be affected. Compared to other inflamed organs in other inflammatory diseases, the relation of affected versus healthy body can be major in IBH. A fourth difficulty concerns the level of exacerbation and chronification (the transition from acute to chronic) during the allergic season, which can be a major factor in IBH because patients such as horses can still scratch intensely, even if painful open wounds are present. A last difficulty concerns the affected organ, more precisely the skin, which is not known to be a target area where injected MSCs would preferably home to in meaningful amounts. In contrast, MSCs have been found in wound tissues several days after transplantation in animal models but their engraftment efficiency ranged from <0.01% when MSCs were intravenously (IV) injected to 3.5% in a study where MSCs were locally applied. After IV injection, in many studies with different types and species of MSCs in different recipient species, MSCs are mostly found in lungs, followed by liver and spleen (Leibacher and Henschler Stem Cell Research & Therapy (2016) 7:7 DOI 10.1186/s13287-015-0271-2). The present ePB-MSCs were shown to preferably home to liver (Beerts C, Brondeel C, Pauwelyn G, Depuydt E, Tack L, Duchateau L, Xu Y, Saunders J H, Peremans K, Spaas JH. Scintigraphic tracking of 99mTechnetium-labelled equine peripheral blood-derived mesenchymal stem cells after intravenous, intramuscular, and subcutaneous injection in healthy dogs. Stem Cell Res Ther. 2021 Jul. 13;12 (1): 393. doi: 10.1186/s13287-021-02457-9. PMID: 34256833; PMCID: PMC8278733.).

In view of at least these unique treatment difficulties the inventors surprisingly found that, even in severe cases of IBH, MSCs could also be effectively used in the treatment of insect-bite hypersensitivity (IBH) in equines.

In a first aspect, the invention relates to MSCs or a pharmaceutical composition comprising an effective amount of MSCs for use in the treatment of insect-bite hypersensitivity (IBH) in equines or as a method for treating IBH in equines or for use in the preparation of a medicament for the treatment of IBH in equines.

Said equine may be any horse-like animal of the Equidae family, preferably of the genus Equus, such as from the species E. caballus (including the myriad domestic strains), E. zebra, E. burchelli, and E. grevyi (zebras) or E. asinus and E. hemionus (wild asses). Said equine is preferably a horse of E. caballus.

The term “effective amount” as used herein refers to the minimum amount or concentration of a compound or composition that is effective to prevent or reduce the symptoms or to ameliorate the condition of a disease. “Effective amount” as used herein can thus be used in the context of prophylactic, metaphylactic and/or therapeutic treatment.

In an embodiment, said MSCs or a pharmaceutical composition comprising an effective amount of MSCs are used for treating IBH mostly caused by an insect of the Ceratopogonidae family. Ceratopogonidae is placed in the infraorder Culicomorpha. It is one of the most diverse families of Diptera, occurring on all continents except Antarctica and in most habitats, including deserts. Traditionally, IBH has been associated with insects of the genus Culicoides; biting midges, also known as no-see-ums; midges; punkies; and sandflies, which belong to the Ceratopogonidae family. As such, in an embodiment, said IBH is caused by Culicoides spp. Culicoides spp. are the most important insects inducing IBH and are also a vector of for instance bluetongue virus and African horse sickness. Culicoides spp. are several millimeters in length and many sub-species (e.g. obsoletus, nubeculosus, punctatus) have been identified. Female Culicoides spp. feed on blood that is needed for egg production, while males feed on nectar. Species causing IBH differ between countries; C. obsoletus was identified as causal agent in British Columbia and the Netherlands, while C. imicola was identified as causal agent in Israel. C. nubeculosus is widespread in Germany, while C. sonorensis is observed frequently in North America.

Culicoides are closely related to Simuliidae (black flies or buffalo gnats), which have also been implicated in IBH. Haematobia irritans (horn flies), Stomoxys calcitrans (stable flies), mosquitos (Culicidae) and less commonly Chrysops (deer flies), Musca (houseflies), Tabanus (horse flies), bees, and wasps may also trigger IBH. Some horses have hypersensitivities to multiple biting insects.

The distribution of IBH skin lesions varies and depends on the feeding preferences of the insect involved. Some Culicoides spp. commonly feed on the mane, tail, dorsum, and ventral midline of the horse, but the face, ears, croup, distal limbs, ventral trunk, and neck can be frequent feeding spots for other Culicoides spp. Black flies tend to feed on the head, ears, and ventral abdomen and may produce a generalized pattern of ventral midline edema. Horn flies typically feed around the umbilicus. Mosquitoes prefer feeding on the lateral aspects of the body, but lesions associated with their bites may be more of a generalized papular urticaria or true hives. Stable flies prefer feeding on the lower legs but have also been known to feed on the ventral abdomen, chest, and back. The lesions in any given IBH horse may change over weeks to months as the specific population of Culicoides spp. or other insects present in the environment changes. Culicoides spp. are typically present in the environment during the summer and during Culicoides exposure, allergic horses develop IBH-related symptoms. Clinical signs start to decrease as soon as Culicoides midges cannot easily bite the skin when winter fur starts to grow (in cold regions), and clinical signs generally start to resolve as soon as midges are not present in environment anymore (typically related to first frost in cold regions), and accordingly allergic horses often appear clinically healthy during the winter.

While feeding, the midges inject a pool of various salivary gland proteins leading to sensitization and allergy in predisposed horses. Transcutaneous absorption and inhalation of desiccated insect parts can also incite the allergic response.

In an embodiment, said treatment is prophylactic, metaphylactic and/or therapeutic.

Ideally, preventive (prophylactic) treatment should be carried out prior to exposure to the allergens. Insect hypersensitivity reactions are seasonal in colder climates but can be nonseasonal in warmer climates in which insects persist year-around. In an embodiment, the prophylactic treatment regimen (timing of administration of the MSCs or composition for use according to the invention) depends on the actual presence of the causative insect in the respective geographical region. For instance, when Culicoides spp. are typically present in the environment in a certain geographical region during the summer, prophylactic treatment should occur prior to the start of summer. In an embodiment, the treatment is metaphylactic, meaning that the equine was exposed to the allergen, but does not show any clinical symptoms yet. In an embodiment, the treatment is therapeutic and improves one or more of the IBH-associated clinical symptoms.

In an embodiment, said treatment is the treatment of one or more IBH-associated clinical symptoms in equines diagnosed with or suffering from IBH.

IBH is characterized initially by papules followed by severe itch (pruritus), which is considered the most prominent clinical symptom during the initial period of IBH manifestation. The itch results in self-inflicted trauma causing further symptoms such as hair loss (alopecia) and skin irritation/dermatitis, wherein the lesions often occur at the head, mane, belly, chest, back or the base of the tail. Lesions are characterized by swelling, scales, crust formation, bleeding, hyperkeratosis, lichenification and wrinkle-building of the skin, depending on the acute or chronic stage. Histologic hallmarks of IBH lesions are thickening of the stratum corneum, epidermis, and dermis, with abundant fibrosis in the latter. Commonly, secondary infections with bacteria, mites, and fungi can cause further local irritation, enhancing lesion formation. In severe cases, IBH can also result in massive weight loss, often while their healthy herd mates gain weight.

In an embodiment, said IBH-associated clinical symptoms are evaluated using an allergy scoring system.

In an embodiment, said IBH-associated clinical symptoms are evaluated using a scoring system according to and/or modified after Wagner (Miller J E, Mann S, Fettelschoss-Gabriel A, Wagner B. Comparison of three clinical scoring systems for Culicoides hypersensitivity in a herd of Icelandic horses. Vet Dermatol. 2019 December;30 (6): 536-e163. doi: 10.1111/vde.12784. Epub 2019 Aug. 22. PMID: 31441172.). In said scoring system pruritus, alopecia and dermatitis (skin irritation) are scored (see table 1 below) and a total IBH symptom score for said three parameters is calculated.

TABLE 1
Wagner scoring system

| CLINICAL SIGN | GRADE | SCORE |
|---|---|---|
| PRURITUS | No mane or tail scratching | 0 |
| | Mild mane and/or tail scratching | 1 |
| | Moderate mane and/or tail | 2 |
| | scratching | |
| | Intense mane and tail scratching | 3 |
| ALOPECIA | None | 0 |
| | Few broken hairs one location | 1 |
| | Several locations with broken hairs | 2 |
| | Moderate hair loss, mane or tail | 3 |
| | Severe hair loss, mane and tail | 4 |
| SKIN IRRITATION | No skin irritation | 0 |
| | Mild dermatitis, one location | 1 |
| | Moderate dermatitis, several | 2 |
| | locations | |
| | Dermatitis with skin lesions | 3 |

In an embodiment a modified Wagner scoring system is used, said modified scoring system is similar to the one described in Table 1, but uses 0.5 steps (thus having additional scoring values 0.5, 1.5, 2.5 and in the case of alopecia also value 3.5) allowing a more precise read-out.

In an embodiment a modified Wagner scoring system is used, wherein pruritus, alopecia and skin irritation are scored in accordance with the Wagner scoring sytem (Table 1) and wherein a score ranging from 0 to 4 for alopecia and ranging from 0 to 3 for pruritus and skin irritation, respectively indicates symptoms being not present (score 0) to being severe (score 4 or 3, respectively). The severity of hair loss is rated by focusing on the broken or lost hair and rough number and size of the areas of alopecia (0; 1 or more for broken and 0; 1 or 2 for lost hair). Broken hair is considered as a mild/moderate symptom, and alopecia as a moderate/severe symptom. The severity of skin irritation is rated by focusing on severity of the dermatitis observed. Swelling, scales and crust are considered as a mild/moderate symptom, and blood and purulence as a moderate/severe symptom.

In an embodiment, said IBH-associated clinical symptoms are evaluated using a scoring system according to and/or modified after Fettelschoss-Gabriel (Fettelschoss-Gabriel A, Fettelschoss V, Olomski F et al. Active vaccination against Interleukin-5 as long-term treatment for insect bite hypersensitivity in horses. Allergy 2018; 142:1,194-1,205). In said scoring system, all locations (tail, mane, belly, flank, face, ear, leg, and the like) at which IBH lesions typically occur are recorded and each location is divided into 3 parts: up, middle, and down. Furthermore, according to the number of lesions, each location is classified as light or strong, depending on the frequency of lesions within the particular area.

Depending on how many parts are affected (up/middle/down) and how many lesions per location are found (light/strong), 1 to 4 points are scored (1 point=one part affected, lesion light; 4 points=all 3 parts affected, lesion strong). Moreover, these locations are classified for 6 further properties: size (diameter), blood (occurrence of bleeding), hair loss, scales, crust, and lichenification/swelling. For all these properties, 1 to 4 points are also scored. Lesion size is divided into 1 point (<0.5 cm), 2 points (0.5≤×<1 cm), 3 points (1≤×<2 cm), and 4 points (22 cm). The “blood” criteria serves to distinguish between intact epidermis (1 point) or whether mild (2 points), moderate (3 points), or severe (4 points) bleeding occurs. Hair loss is divided into mild (1 point), moderate (2 points), severe (3 points), and absence of hair (4 points). Scales are divided into none (1 point), tiny and few (2 points), moderate and midsized (3 points), and many and big (4 points) scales. Crust is divided into none (1 point), tiny (2 points), half (3 points), and total (4 points). Lichenification and/or swelling are divided into none (1 point), mild (2 points), moderate (3 points), and severe (4 points). Additionally, if the sheath or udder is swollen, a minimum of 5 or a maximum of 20 points are scored: grade 1 (5 points), grade 2 (10 points), grade 3 (15 points), and grade 4 (20 points). Finally, all points are summed to produce the IBH symptom (or lesion) score.

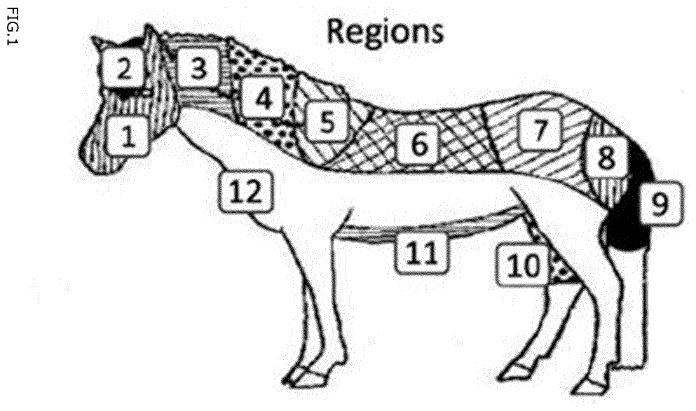

In an embodiment, said IBH-associated clinical symptoms are evaluated using a scoring system according to and modified after Geiben (Geiben T. Studies on summer eczema and on the influence of the immunomodulator Baypamun N® on type I allergy in horses. Doctoral thesis 2003. Veterinary School Hannover, Germany —https://dnb.info/969238762/34 Accessed Mar. 7, 2019). In said scoring system twelve regions (see FIG. 1) are investigated regarding seven symptoms (wheals, papules, broken hairs, alopecia, crusts/scabing, blood/exudate and purulence) giving a score of 0 (not present), 1 (very mild), 2 (mild), 3 (mild to moderate), 4 (moderate), 5 (moderate to severe), or 6 (severe).

In an embodiment, the treatment results in a decreased or annihilated IBH-associated inflammatory response in the equine(s) receiving the treatment. In an embodiment, said decrease in IBH-associated inflammatory response refers to a decreased response measured in equines receiving the MSCs or pharmaceutical composition comprising MSCs of the invention of at least 10%, preferably at least 20%, more preferably at least 30%, more preferably at least 40%, more preferably at least 50%, more preferably at least 60%, more preferably at least 70%, more preferably at least 80%, compared to the IBH-associated inflammatory response measured in a non-treated or placebo treated control group of the same species. In an embodiment, said decrease in IBH-associated inflammatory response refers to a decreased response measured in equines receiving the MSCs or pharmaceutical composition comprising MSCs of the invention of at least 10%, preferably at least 20%, more preferably at least 30%, more preferably at least 40%, more preferably at least 50%, more preferably at least 60%, more preferably at least 70%, more preferably at least 80%, compared to the IBH-associated inflammatory response measured in said equines when not receiving said MSCs or said pharmaceutical composition comprising MSCs. Said comparison can comprise a comparison for the entire treatment group or for each individual equine between two (preferably consecutive) IBH seasons, wherein during a first IBH season the equine(s) did not receive the MSCs or the composition for use according to the current invention and during a second IBH season the equine(s) did receive the MSCs or the composition for use according to the current invention.

An “IBH season” as used herein refers to a certain period in a certain calendar year wherein the causative insect(s) are/is present and elicit(s) symptoms in sensitised equines. In an embodiment, an average of multiple seasons can be calculated and used in the comparison. In an embodiment, a comparison for the entire treatment group or for each individual equine is made between two (preferably consecutive) IBH seasons, wherein during a first IBH season the equine(s) did not receive the MSCs or the composition for use according to the current invention and during a second IBH season the equine(s) did receive the MSCs or the composition for use according to the current invention. “First” and “second” do not refer to a chronological order, the “first IBH season” can thus refer to a season (or average of seasons) occurring before or after the second IBH season.

A decreased inflammatory response can be reflected by a decrease in the IBH-associated clinical symptoms, such as a decreased pruritus and/or decreased alopecia and/or decreased dermatitis and can refer to a decrease in the severity and the number of the lesions.

In an embodiment, the treatment results in an improvement in one or more of the IBH-associated clinical symptoms, such as pruritus, alopecia, broken hairs, dermatitis (acute or chronic lesions), weight loss, bulges/lichenification, wheals, papules, crusts/scabing, blood/exudate, purulence or combinations thereof.

In an embodiment, the treatment results in an improvement in one or more of the IBH-associated clinical symptoms consisting of: pruritus, alopecia, dermatitis (acute or chronic lesions), weight loss or combinations thereof.

In an embodiment, the treatment results in a decreased pruritus and/or decreased alopecia and/or decreased dermatitis (including a decrease in bulges/lichenification, wheals, papules, crusts/scabing, blood/exudate and/or purulence) and/or decreased weight loss measured in equines receiving the MSCs or pharmaceutical composition comprising MSCs of the invention in comparison to a non-treated or placebo treated control group of the same species or in comparison to said equines when not receiving said MSCs or said pharmaceutical composition comprising MSCs. In an embodiment, said decrease refers to a decrease of at least 10%, preferably at least 20%, more preferably at least 30%, more preferably at least 40%, more preferably at least 50%, more preferably at least 60%, more preferably at least 70%, more preferably at least 80%, compared to a non-treated or placebo treated control group of the same species or in comparison to said equines when not receiving said MSCs or said pharmaceutical composition comprising MSCs. As discussed above, said comparison can be made between two (preferably consecutive) IBH seasons, wherein during a first IBH season the equine(s) did not receive the MSCs or the composition for use according to the current invention and during a second IBH season the equine(s) did receive the MSCs or the composition for use according to the current invention treatment.

In an embodiment, said decreased pruritus and/or decreased alopecia and/or decreased dermatitis is measured using a scoring system, such as the Wagner scoring system discussed above. In an embodiment, said decreased pruritus and/or decreased alopecia and/or decreased dermatitis refers to a decrease in score measured in equines receiving the MSCs or pharmaceutical composition comprising MSCs of the invention of at least 10%, preferably at least 20%, more preferably at least 30%, more preferably at least 40%, more preferably at least 50%, more preferably at least 60%, more preferably at least 70%, more preferably at least 80%, compared to the score measured in a non-treated or placebo treated control group of the same species or compared to the score measured in said equines when not receiving said MSCs or said pharmaceutical composition comprising MSCs.

As mentioned above, various IBH symptoms can be measured by means of allergy scoring systems and an overall IBH symptom score can be assigned to a certain equine or to a group of equines.

In an embodiment, the treatment results in a decreased IBH symptom score in equines receiving the MSCs or the pharmaceutical composition comprising an effective amount of MSCs for use according to the current invention in comparison to a non-treated or placebo treated control group of the same species.

In an embodiment, a A average IBH symptom score is calculated for placebo-treated and MSC-treated horses as follows. IBH symptom scores for all horses are recorded weekly (or bi-weekly) during the observation period within each IBH season. For each horse, the average IBH symptom score is calculated per season. The A average IBH symptom score is defined by subtracting the pretreatment season average IBH symptom score by the treatment season average IBH symptom score. The average of the resulting differences is calculated and is reported as A mean average IBH symptom score.

In an embodiment, the treatment results in a decreased IBH symptom score in equines receiving the MSCs or the pharmaceutical composition comprising an effective amount of MSCs for use according to the current invention in comparison to the IBH symptom score measured in said equines when not receiving said MSCs or said pharmaceutical composition comprising MSCs.

In an embodiment, IBH-associated clinical symptoms are measured using an allergy scoring system, wherein administration of one or more doses of the MSCs or the pharmaceutical composition comprising MSCs to an equine diagnosed with or suffering from IBH results in a relative decrease of at least 10%, preferably at least 20%, more preferably at least 30%, more preferably at least 40%, more preferably at least 50%, more preferably at least 60%, more preferably at least 70%, more preferably at least 80%, in the measured allergy score compared to the measured allergy score of said equine when not administered said MSCs or said pharmaceutical composition comprising MSCs.

In an embodiment, said comparison comprises a comparison of the % change in area under the curve (AUC) for the entire treatment group or for each individual equine between two (preferably consecutive) IBH seasons, wherein during a first IBH season the equine(s) did not receive the MSCs or the composition for use according to the current invention and during a second IBH season the equine(s) did receive the MSCs or the composition for use according to the current invention treatment. In an embodiment, 30% and 50% decrease values are estimates for moderate and good efficacy, respectively.

In an embodiment, said MSCs for use are native. Such native MSCs have not first in vitro been exposed to a stimulating agent, such as inflammatory mediators or an inflammatory environment. The use of native MSCs is sometimes a favorable option as they allow the production of a ready-to-use product, with minimum manufacturing and handling, thereby lowering cost of production and having less supply risk, batch variability risk and less risk for contamination. The majority of previous published studies concerning MSCs are using autologous or allogeneic MSCs derived from adipose tissue or bone marrow (BM).

The MSCs for use according to the present invention may originate from various tissues or body fluids, in particular from blood, BM, fat tissue or amniotic tissue. Bone marrow harvesting of MSCs has been reportedly associated with haemorrhage, chronic pain, neurovascular injury, and even death. Adipose tissue as a source for MSCs is regarded as a safer option. However, harvesting of MSCs from adipose tissue still requires an incision in the donor animal, hence this is still an invasive procedure. MSCs derived from blood show similar morphology as MSCs derived from bone marrow and adipose tissue. As a consequence, by preference, the MSCs originate from blood, including but not limited to umbilical cord blood and peripheral blood. More preferably, the MSCs originate from peripheral blood. Blood is not only a non-invasive and painless source, but also simple and safe to collect and, consequently, easily accessible and prone to less complications afterwards.

The MSCs or blood comprising MSCs may originate from all mammals, including, but not limited to, humans, domestic and farm animals, zoo animals, sport animals, pet animals, companion animals and experimental animals, such as, for example, mice, rats, rabbits, dogs, cats, cows, horses, pigs and primates, e.g., monkeys and apes; especially horse, human, cat, dogs, rodents, etc. In a preferred embodiment, said MSCs are equine-derived. In particular, said MSCs may be derived from peripheral blood, preferably equine peripheral blood, which allows multiple MSC collections per year with minimal discomfort or morbidities for the donor animal.

In some cases, the use of allogeneic MSCs is a more favorable option as they offer a stringent selection of healthy and high-quality stem cell donors. They allow the production of a ready-to-use product, avoiding the invasive harvesting and time-consuming cultivation of MSCs from each individual patient.

Therefore, in a particular embodiment the MSCs of the current invention may be used for allogeneic administration to a subject. As already indicated, allogeneic use allows a better control of the quality of the MSCs, as different donors may be screened, and the optimal donors may be selected. The latter is indispensable in view of preparing functional MSCs. This is in contrast to autologous use of MSCs, as in this case, quality of the cells is more difficult to be ensured. Nonetheless, autologous use may have his benefits as well. In the case of autologous use, blood MSCs are isolated, for which blood from a donor was used who was later also recipient of the isolated MSCs. In the case of allogeneic use, blood is used from donors in which the donor is preferably of the same family, gender or ethnicity as the recipient of the MSCs isolated from the blood of donors. In particular, these donors will be tested on common current transmittable diseases or pathologies, in order to avoid the risk of horizontal transmission of these pathologies or diseases through the stem cells. Preferably, the donors/donor animals are kept in quarantine. When using donor horses they can be, for example tested for the following pathologies, viruses or parasites: equine infectious anemia (EIA), equine rhinopneumonitis (EHV-1, EHV-4), equine viral arteritis (EVA), West Nile virus (WNV), African horse Sickness (AHS), dourine (Trypanosoma), equine piroplasmosis, glanders (malleus, glanders), equine influenza, Lyme borreliosis (LB) (Borrelia burgdorferi, Lyme disease).

The MSCs of current invention may be derived by any standard protocol known in the art. In an embodiment, said MSCs may be obtained via a method wherein the MSCs are isolated from blood or a blood phase and wherein said cells are cultured and expanded in a basal medium, preferably a low glucose medium.

Basal medium formulation as known in the art include, but are not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's 10 MB 752/1 or Williams Medium E, and modifications and/or combinations thereof. Compositions of the above basal media are generally known in the art and it is within the skill of one in the art to modify or modulate concentrations of media and/or media supplements as necessary for the cells cultured. A preferred basal medium formulation may be one of those available commercially such as DMEM, which are reported to sustain in vitro culture of MSCs, and including a mixture of growth factors for their appropriate growth, proliferation, maintenance of desired markers and/or biological activity, or long-term storage.

Such basal media formulations contain ingredients necessary for mammal cell development, which are known per se. By means of illustration and not limitation, these ingredients may include inorganic salts (in particular salts containing Na, K, Mg, Ca, CI, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g. glucose, pyruvate, e.g., sodium pyruvate, acetate, e.g., sodium acetate), etc. It will also be apparent that many media are available as low-glucose formulations with or without sodium pyruvate.

Method for isolating MSCs from blood or a blood phase and culturing and expanding said cells are known in the art and for instance described in WO2014053418 or WO2014053420.

In an embodiment, such method for isolating MSCs from blood or a blood phase and culturing and expanding said cells in a low glucose medium may comprise the following steps:
a) the collection of one or more blood samples from donors, in a sample vial, coated with an anti-coagulant;
b) centrifuging the blood samples to obtain a 3-phase distribution, consisting of a plasma-phase, buffy coat, and erythrocytes phase;
c) collecting the buffy coat and loading it on a density gradient;
d) collecting of the blood-inter-phase obtained from the density gradient of step c);
e) isolating of MSCs from the blood-inter-phase by centrifugation;
f) seeding between 2.5×10⁵/cm² and 5×10⁵/cm² MSCs in culture and keeping them in a low glucose growth medium supplemented with dexamethasone, antibiotics and serum.

In an embodiment, anticoagulants may be supplemented to the MSCs. Non-limiting examples are EDTA or heparin.

The number of seeding is crucial to ultimately obtain a pure and viable population MSCs at an acceptable concentration, as a too dense seeding will lead to massive cell death during expansion and a non-homogenous population of MSCs and a too dispersed seeding will result in little or no colony formation of MSCs, so that expansion is not or hardly possible, or it will take too much time. In both cases the viability of the cells will be negatively influenced.

In a preferred embodiment of current invention, the MSCs have a high cell viability, wherein at least 90%, more preferably at least 95%, most preferably 100% of said cells are viable.

The blood-interphase is the source of blood mononuclear cells (BMCs) comprising monocytes, lymphocytes, and MSCs. By preference, the lymphocytes are washed away at 37° C., while the monocytes die within 2 weeks in the absence of cytokines necessary to keep them alive. In this way, the MSCs are purified. The isolation of the MSCs from the blood-inter-phase is preferably done by means of centrifugation of the blood-inter-phase, after which the cell pellet is washed at least once with a suitable buffer, such as a phosphate buffer.

In a further embodiment the MSCs of current invention are negative for monocytes and macrophages, both within a range between 0% and 7.5%.

In particular, the mesenchymal cells are kept at least 2 weeks in growth medium. Preferably, growth medium with 1% dexamethasone is used, as the specific characteristics of the MSCs are kept in said medium.

Following a minimum period of 2 weeks (14 days), preferably 3 weeks (21 days) MSC colonies will become visible in the culture bottles. In a subsequent step g) at least 6×10³ stem cells/cm² are transferred to an expansion medium containing low glucose, serum and antibiotics for the purpose of expanding the MSCs. Preferably, the expansion of the MSCs will occur in minimal five cell passages. In this way sufficient cells can be obtained. Preferably, the cells are split at 70% to 80% confluency. The MSCs can be maintained up to 50 passages in culture. After this the risk of loss in vitality, senescence or mutation formation occurs.

In a further embodiment, the population doubling time (PDT) between each passage during expansion of the MSCs should be between 0.7 and 3 days after trypsinization. Said PDT between each passage during expansion of the MSCs is preferably between 0.7 and 2.5 days after trypsinization.

In a preferred embodiment, the MSCs for use according to the invention have a spindle-shaped morphology. The morphological characterization of the MSCs of current invention classifies the cell as an elongated, fibroblast-like, spindle-shaped cell. This type of cell is distinct form other populations of MSCs with small self-renewing cells which reveal mostly a triangular or star-like cell shape and populations of MSCs with a large, cuboidal or flattened pattern with a prominent nucleus. The selection of MSCs with this specific morphological characteristic along with the biological markers enables the person skilled in the art to isolate the MSCs of current invention. A morphological analysis of cells can easily be performed by a person skilled in the art using phasecontrast microscopy. Besides, the size and granularity of MSCs can be evaluated using forward and side scatter diagram in flow cytometry or other techniques known by a person skilled in the art.

In another or further preferred embodiment, the MSCs have a suspension diameter between 10 μm and 100 μm. The MSCs for use of current invention have been selected based on size/suspension diameter. By preference, the MSCs have a cell size between 10 to 100 μm, more preferably between 15 and 80 μm, more preferably 20 and 75 μm, more preferably between 25 and 50 μm. Preferably, the selection of cells based on cell size occurs by a filtration step. For instance, MSCs with a cell concentration ranging between 103 to 107 MSCs per ml, wherein said cells are preferably diluted in low glucose DMEM medium, are selected by size by means of a filter system, wherein the cells are run through a double filtration step using a 40 μm filter. Double or multiple filtration steps are preferred. The latter provides for a high population of single cells and avoids the presence of cell aggregates. Such cell aggregates may cause cell death during the preservation of the cells by freezing and may all have an impact on further downstream applications of the cells. For instance, cell aggregates may higher the risk of the occurrence of a capillary embolism when administered intravenously. In an embodiment, said composition comprises at least 75%, more preferably at least 80%, even more preferably at least 85%, most preferably at least 90% of single cells and whereby said single cells have a suspension diameter of between 10 μm and 100 μm, more preferably between 15 μm and 80 μm, more preferably between 20 μm and 75 μm, more preferably between 25 μm and 50 μm. As previously mentioned, the diameter of the cells as well as their single-cell nature is crucial for any downstream application, e.g. intravenous administration, and for the vitality of the cells.

In an embodiment, the MSCs for use according to the present invention are formulated in a sterile liquid for administration to a subject.

In a preferred embodiment, the MSCs for use according to the present invention are formulated for administration in a subject by means of intravenous injection or infusion.

In an embodiment, the volume of the composition (and the amount of MSCs) which is administered per injection or infusion to a patient is adapted in accordance with the patient's body weight. In another embodiment, the volume of the composition (and the amount of MSCs) which is administered per injection or infusion to a patient is adapted in accordance with body surface of the patient. In yet another embodiment, the volume of the composition (and the amount of MSCs) which is administered per injection or infusion to a patient is adapted in accordance with affected surface area of the patient. In yet another embodiment, a dose of 10⁵-108 MSCs per patient, preferably 106 to 108 MSCs, more preferably 107-108 MSCs is administered.

In an embodiment, with each intravenous injection or infusion, an effective amount of MSCs is administered, preferably each injection or infusion comprises a dose of between 10⁵ to 10⁸ of said MSCs. Preferably, the MSCs are administered through intravenous injection.

In an embodiment, a single dose is administered. In another embodiment, multiple doses are administered with each dose being administered at different time points. In an embodiment, when multiple doses are administered, administrations are done in time intervals of every 1 to 8 weeks, preferably every 2 to 6 weeks, such as every 2 weeks. In an embodiment, 1 to 15 administrations, preferably 1 to 6 administrations, such as 4 administrations are done in total. In a preferred embodiment, 3 administrations are done in total. Preferably each dose is administered 2-4 weeks after the previous dose. In a preferred embodiment, each dose is administered 4 weeks after the previous dose. Preferably the last dose is administered within 9 months, more preferably within 6 months after the first dose. Preferably the last dose is administered within 6 months after the first dose. In a preferred embodiment, 3 administrations are done in total, each dose being administered 4 weeks after the previous dose. In an embodiment, the treatment scheme (number of doses, time interval between consecutive administrations, administration of the first dose, administration of the last dose and/or amount of MSCs administered) depends on the season length and/or, related thereto, the actual presence of the causative insect in the respective geographical region. In an embodiment, timing of administration of the first dose is dependent on the activity of the causative insect in the respective geographical region. In an embodiment, timing of administration of the last dose is dependent on the activity of the causative insect in the respective geographical region. In an embodiment, timing of administration of the first and last dose is dependent on the activity of the causative insect in the respective geographical region. In an embodiment, the last dose is administered in midsummer.

In an embodiment, when multiple doses are administered, each administered dose is identical, meaning each dose comprises approximately the same amount of MSCs. In another embodiment, when multiple doses are administered, not each administered dose is identical, meaning each dose does not comprise approximately the same amount of MSCs.

In an embodiment, the number of doses depends on the severity of the disease. In an embodiment, a single dose is administered to patients presenting only mild symptoms, whereas multiple doses are administered to patients presenting severe symptoms.

In an embodiment, a particularly effective treatment is achieved by a dosing regimen comprising at least two dosages, preferably at least 3 dosages of the MSCs for use or the pharmaceutical composition for use as described above in any of the embodiments.

Therefore, a further embodiment relates to a pharmaceutical composition for use in the treatment of IBH, wherein:
the treatment comprises a step of administering, preferably intravenously, a first amount of said composition comprising a total dose of between 10⁷-10⁸ MSCs per patient, and
the treatment further comprises a step of administering, preferably intravenously, a second amount of said composition, said second amount comprising a second total dose of between 10⁷-10⁸ MSCs per patient,
the treatment further comprises a step of administering, preferably intravenously, a third amount of said composition, said third amount comprising a third total dose of between 10⁷-10⁸ MSCs per patient,
   wherein said second dose and third dose are administered 6 days after the previous dose, 7 days (1 week) after the previous dose, 2 weeks after the previous dose, 3 weeks after the previous dose, 4 weeks after the previous dose, 5 weeks after the previous dose, 6 weeks after the previous dose, 7 weeks after the previous dose, 8 weeks after the previous dose, 3 months after the previous dose, 6 months, 9 months after the previous dose, and/or 1 year after the previous dose. Preferably each dose is administered 2-4 weeks after the previous dose. Preferably the last dose is administered within 12 months, preferably within 9 months, more preferably within 6 months after the first dose.

In an embodiment, said first, second and third total dose are identical. In another embodiment, said first, second and third total dose are not identical. In an embodiment, said second dose is identical to the first dose. In another embodiment, said second dose is lower than the first dose. In yet another embodiment, said second dose is higher than the first dose. In an embodiment, said second dose is identical to the third dose. In another embodiment, said second dose is lower than the third dose. In yet another embodiment, said second dose is higher than the third dose. In an embodiment, said third dose is identical to the first dose. In another embodiment, said third dose is lower than the first dose. In yet another embodiment, said third dose is higher than the first dose.

In an embodiment, one dose of said composition has a volume of about 1 to 100 ml, preferably of about 5 to 75 ml, preferably of about 5 to 50 ml, preferably of about 8 to 20 ml, most preferably of about 10 ml. In another or further embodiment, one dosage of said composition has a volume of maximally about 100 ml, preferably maximally about 50 ml, more preferably maximally about 25 ml, more preferably maximally about 20 ml, most preferably said volume is about 10 ml. This amount is suitable for intravenous administration. Said dose may be formulated in a vial or in a pre-filled syringe.

In an embodiment, the MSCs for use of the present invention may be characterized by the presence of/are measured positive for one or more of the following markers CD29, CD44, CD90, CD105, vimentin, fibronectin, Ki67, CK18 or any combination thereof. In a further embodiment, the MSCs for use of current invention may be characterized by the presence of mesenchymal markers CD29, CD44 and CD90. By means of the latter, the purity of the obtained MSCs can be analyzed, and the percentage of MSCs can be determined. CD29 is a cell surface receptor encoded by the integrin beta 1 gene, wherein the receptor forms complexes with other proteins to regulating physiological activities upon binding of ligands. The CD44 antigen is a cell surface glycoprotein involved in cell-cell interactions, cell adhesion and migration. In addition, is CD44 a receptor for hyaluronic acid and can also interact with other ligands such as osteopontin, collagens and matrix metalloproteinases (MMPs). The CD90 antigen is a conserved cell surface protein considered as a marker for stem cells, like MSCs. The MSCs of current invention being triple positive for CD29/CD44/CD90 enables the person skilled in the art for a fast and unambiguous selection of the MSCs and provides the MSCs biological properties which are of interest for further downstream applications.

In an embodiment, the MSCs for use of the current invention are characterized by the absence of/measure negative for Major Histocompatibility Complex (MHC) class II molecules, preferably all currently known MHC Class II molecules, classifying the cell as a cell that can be used in cellular therapy for mammalians, such as equine cellular therapy. Even when the MSCs are partly differentiated, the MSCs remain negative for MHC class II molecules. Detecting presence or absence, and quantifying the expression of MHC II molecules can be performed using flow cytometry.

In another and further embodiment the MSCs measure negative for CD45 antigen, a marker for hematopoietic cells.

In an embodiment, the MSCs measure negative for both MHC class II molecules and CD45.

In a particularly preferred embodiment, the MSCs for use of the current invention measure positive for mesenchymal markers CD29, CD44 and CD90 and measure negative for MHC class II molecules and CD45.

MSCs in general express MHC Class I antigen on their surface. In a particular embodiment the MSCs for use of current invention have a low or undetectable level of the MHC Class I marker. In a most preferred embodiment said MSCs measure negative for MHC Class II markers and have a low or undetectable level of MHC Class I marker, wherein said cell exhibits an extremely low immunogenic phenotype.

For the sake of the current invention, said low level should be understood as less than 25%, more preferably less than 15% of the total cells expressing said MHC I or MHC II. Detecting presence or absence, and quantifying the expression of MHC I and MHC II molecules can be performed using flow cytometry.

In a preferred embodiment the MSCs for use of the current invention measures:
positive for mesenchymal markers CD29, CD44 and CD90;
positive for one or more markers comprised in the group consisting of vimentin, fibronectin, Ki67, or a combination thereof;
negative for MHC class II molecules;
negative for hematopoietic marker CD45; and
preferably have a low or undetectable level of MHC Class I molecules, wherein said low level should be understood as less than 25%, more preferably less than 15% of the total cells expressing MHC I.

These immunological properties of the MSCs limit the ability of the recipient immune system to recognize and reject cells, preferably allogeneic cells, following cellular transplantation. The production of factors by MSCs, that modulate the immune response together with their ability to differentiate into appropriate cell types under local stimuli make them desirable stem cells for cellular therapy.

In general, any technology for identifying and characterizing cellular markers for a specific cell type (e.g. mesenchymal, hepatic, hematopoietic, epithelial, endothelial markers) or having a specific localization (e.g. intracellular, on cell surface, or secreted) that are published in the literature may be considered appropriate for characterizing MSCs. Such technologies may be grouped in two categories: those that allow maintaining cell integrity during the analysis, and those based on extracts (comprising proteins, nucleic acids, membranes, etc.) that are generated using such cells. Among the technologies for identifying such markers and measuring them as being positive or negative, immunocytochemistry or analysis of cell culture media are preferred since these allow marker detection even with the low amount of cells, without destroying them (as it would be in the case of Western Blot or Flow Cytometry).

Relevant biological features of the MSCs can be identified by using technologies such as flow cytometry, immunocytochemistry, mass spectrometry, gel electrophoresis, an immunoassay (e.g. immunoblot, Western blot, immunoprecipitation, ELISA), nucleic acid amplification (e.g. real time RT-PCR), enzymatic activity, omics-technologies (proteomics, lipidomics, glycomics, translatomics, transcriptomics, metabolomics), in vitro tests (such as following the MLR assay principle, also known as a proliferation assay when mixing lymphocytes with MSCs) and/or other biological activity.

In an embodiment, said MSCs secrete immunomodulatory prostaglandin E2 cytokine when present in an inflammatory environment or condition.

Inflammatory environments or conditions are characterized by the recruitment of immune cells of the blood. Inflammatory mediators include prostaglandins, inflammatory cytokines such as IL-1β, TNF-α, IL-6 and IL-15, chemokines such as IL-8 and other inflammatory proteins like TNF-α, IFN-γ. These mediators are primarily produced by monocytes, macrophages, T-cells, B-cells to recruit leukocytes at the site of inflammation and subsequently stimulate a complex network of stimulatory and inhibitory interactions to simultaneously destruct and heal the tissue from the inflammatory process.

Prostaglandin E2 (PgE₂) is a subtype of the prostaglandin family. PgE₂ is synthesized from arachidonic acid (AA) released from membrane phospholipids through sequential enzymatic reactions. Cyclooxygenase-2 (COX-2), known as prostaglandin-endoperoxidase synthase, converts AA to prostaglandin H2 (PgH2), and PgE₂ synthase isomerizes PgH2 to PgE₂. As a rate-limiting enzyme, COX-2 controls PgE₂ synthesis in response to physiological conditions, including stimulation by growth factors, inflammatory cytokines and tumor promoters.

In a particular embodiment, said MSCs present in an inflammatory environment secrete the soluble immune factor prostaglandin E2 (PgE₂) in a concentration ranging between 103 to 106 picogram per ml to induce or stimulate MSC-regulated immunosuppression.

The PgE₂ secretion of the MSCs in those specific concentration ranges stimulates anti-inflammatory processes in vitro and together with their ability to differentiate into appropriate cell types makes them desirable for cellular transplantation.

In an embodiment, said MSCs have an increased secretion of at least one of the molecules chosen of IL-6, IL-10, TGF-β, NO (nitric oxide), or a combination thereof; and/or a decreased secretion of IL-1 when present in an inflammatory environment or condition and compared to a cell having the same characteristics but not being subjected to said inflammatory environment or condition.

In an embodiment, the MSCs have an increased secretion of PgE₂ in combination with two or more of the abovementioned factors.

PgE₂, IL-6, IL-10, TGF-β and NO help suppressing the proliferation and function of major immune cell populations like T cells and B cells. In addition, the MSCs express low levels of MHC class I molecules and/or are negative for MHC class II molecules on their surface, escaping immunogenic reactions. In addition, the MSCs of current can suppress the proliferation of white blood cells by their increased secretion of abovementioned factors, once again helping to avoid immunogenic reactions of the host.

In an embodiment, said MSCs stimulate the expression of PgE₂, IL-6, IL-10, NO, or a combination thereof when in the presence of PBMCs and/or suppress the secretion of TNF-α, IFN-γ, IL-1, TGF-β, IL-13 or a combination thereof when in the presence of PBMCs.

In the inflammatory environment the MSCs secrete multiple factors that modulate the immune response of the host. In addition, the MSCs have the stimulatory effect to induce or stimulate the secretion of one or more factors selected from the group consisting of PgE₂, IL-6, IL-10, NO, or a combination thereof. Next to the stimulatory effect of the MSCs on the PBMCs in an inflammatory environment, the MSCs also have a suppressive effect on the secretion of the PBMCs, resulting in a decrease of one or more factors selected from the group consisting of TNF-α, IFN-γ, IL-1, TGF-β, IL-13, or a combination thereof. The MSCs have a regulatory effect in the inflammatory environment, making them useful in the treatment of all sorts of diseases, particularly disorders of the immune system.

In an embodiment, the current invention relates to MSCs or a pharmaceutical composition comprising an effective amount of MSCs for use as described above, wherein:
a) said MSCs measure negative for MHC class II molecules and/or CD45, and/or
b) said MSCs measure positive for mesenchymal markers CD29, CD44 and CD90 and measure negative for MHC class II molecules and CD45, and/or
c) said MSCs secrete immunomodulatory prostaglandin E2 cytokine when present in an inflammatory environment or condition, and/or
d) said MSCs have an increased secretion of at least one of the molecules chosen of IL-6, IL-10, TGF-β, NO, or a combination thereof, and/or
e) said MSCs have a decreased secretion of IL-1 when present in an inflammatory environment or condition and compared to a cell having the same characteristics but not being subjected to said inflammatory environment or condition, and/or
f) said MSCs stimulate the expression of PgE₂, IL-6, IL-10, NO, or a combination thereof when in the presence of PBMCs and/or suppress the secretion of TNF-α, IFN-γ, IL-1, TGF-β, IL-13 or a combination thereof when in the presence of PBMCs.

The MSCs or the pharmaceutical composition comprising MSCs for use according to the current invention, will by preference be frozen in order to allow long-time storage of the MSCs or of the composition. Preferably the MSCs or composition will be frozen at low and constant temperature, such as a temperature below −20° C. These conditions allow a save storage of the MSCs or composition, and enable the MSCs to keep their biological and morphological characteristics, as well as their high cell viability during storage and once thawed.

In a more preferred embodiment, the MSCs or the pharmaceutical composition comprising MSCs for use according to the current invention can be stored for at least 6 months at a maximum temperature of −80° C., optionally in liquid nitrogen. A crucial factor in the freezing of the MSCs is a cryogenic medium, in particular comprising DMSO. DMSO prevents ice crystal formation in the medium during the freezing process, but may be toxic to the cells in high concentrations. In a preferred embodiment, the concentration of DMSO comprises up to 20%, more preferably up to 15%, more preferably the concentration of DMSO in the cryogen comprises 10%. The cryogenic medium further comprises low-glucose medium such as low glucose DMEM (Dulbecco's Modified Eagle Medium).

Afterwards, the MSCs or the pharmaceutical composition comprising MSCs for use according to the current invention are preferably thawed before administration at a temperature around room temperature, preferably at a temperature between 20° C. and 37° C., more preferably at a temperature between 25° C. and 37° C., and in a time span of maximal 20 minutes, preferably maximal 10 minutes, more preferably maximal 5 minutes.

Furthermore, the MSCs or composition is preferably administered within 30 minutes after thawing, in order to safeguard the vitality of the MSCs.

The invention is further described by the following non-limiting examples which further illustrate the invention, and are not intended to, nor should they be interpreted to, limit the scope of the invention.

### EXAMPLES

The present invention will now be further exemplified with reference to the following examples. The present invention is in no way limited to the given examples.

### Example 1

Set-up:

A previous study investigating the safety profile of IV administration of the equine peripheral blood-derived mesenchymal stem cells in healthy horses showed no adverse event reactions to treatment. The objective of this study was to evaluate the therapeutic potential of equine peripheral blood-derived mesenchymal stem cells (ePB-MSCs, the investigational product (IVP)) in horses suffering from insect bite hypersensitivity (IBH) employing native (unprimed) stem cells.

Efficacy parameters included severity of three clinical signs of IBH, ie pruritus, alopecia and dermatitis. As up to seven years of disease history of the horses are available, the pattern of clinical sign expression in 2021 served as comparison, ie negative control and previous years served as supportive data. As such, results in treated affected horses were compared to IBH severity as documented in 2021 and previous years. More specifically, the study was based on the 2021 characterization data of the IBH-affected horses (clinical allergy scoring by Wagner score). Clinical signs of the treatment year 2022 were compared to the untreated year 2021. Weather differences could favor or limit the proliferation and activity of the biting midges. In consequence, weather could influence the expression of clinical signs. To understand the impact of weather in the study, the parameters of minimum and maximum daily temperature, daily humidity and daily precipitation were monitored as controls and showed high level of concordance, hence confirming equivalent biting insect pressure.

The horses received 4 IV injections, two to four weeks apart. The animals are of similar weight and stem cell dosing follows the principle of vaccines, meaning that there was no weight adjustment for this exploratory study. The first injection comprised a dose of 15×10⁶ MSCs and occurred in Week 20 (May 17, 2022), the second injection comprised a dose of 15×10⁶ MSCs and occurred in Week 22 (May 31, 2022), the third injection comprised a dose of 40×10⁶ MSCs and occurred in

Week 24 (Jun. 13, 2022) and the last injection comprised a dose of 60×10⁶ MSCs and occurred in Week 28 (Jul. 11, 2022).

Clinical scores were evaluated in two ways:

1) Comparison of the % change in area under the curve (AUC) for the entire treatment group and for each individual horse between 2022 and 2021, wherein 30% and 50% decrease values are estimates for moderate and major efficacy, respectively. Area under the curve (AUC) was calculated covering the whole season (weeks 21-46).

Isolation and Cultivation of ePB-MSCs

According to previously described methods, the ePB-MSCs are isolated from venous blood collected from the vena jugularis of one donor horse. Prior to cultivation of the ePB-MSCs, serum is tested for the presence of multiple transmittable diseases as described by Broeckx et al. 2012. Subsequently the stem cells are cultivated in a Good manufacturing practice (GMP)-certified production site according to GMP-guidelines until passage (P) 5 and characterized on viability, morphology, presence of cell surface markers and population doubling time. Evaluation of the presence (Cluster of Differentiation CD29, CD44 and CD90) and absence (Major Histocompatibility Complex (MHC) II and CD45) of specific cell surface markers is accomplished by using flow cytometry as previously described (Spaas et al., 2013). However, the detailed expression and secretion pattern has been previously described in WO 2020/182935. The cell viability is assessed using trypan blue. Afterwards, the cells are further cultivated until P10, trypsinized and resuspended at a final concentration of 300.000 cells/mL in Dulbecco's Modified Eagle Medium (DMEM) low glucose with 10% dimethylsulfoxide (DMSO). The ePB-MSCs are stored at −80° C. in cryovials until further use. Sterility of the final product is tested by the absence of aerobic bacteria, anaerobic bacteria, fungi, endotoxins and mycoplasma. Treatment was conducted by injection in the jugular vein, using disposable syringes and needles. The prepared vials were thawed in the laboratory and the ePB-MSCs were resuspended in 1 ml sterile physiological saline in glass vials, so that cell viability was maintained during transport to the stable. For final injection, the glass vials were filled to 10 ml with physiological saline.

### Results

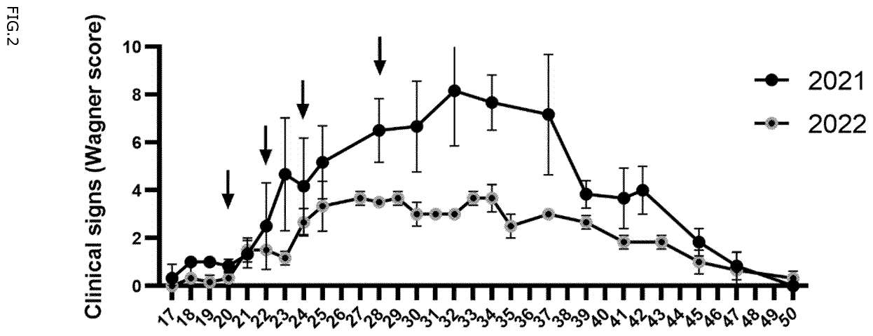

FIG. 2 depicts the clinical score for the entire group of horses (mean and range, n=3), for the same period of time in 2021 (the horses did not receive the IVP, negative control) and in 2022 (the horses received the IVP). Area under the curve (AUC) was calculated across the IBH season for weeks 21-46 (see Table 2 below). Comparison of the % change in area under the curve (AUC) for the entire treatment group across season (for weeks 21-46), shows a change of −50% from 2021 to 2022, indicating efficacy of the IVP. Furthermore, as described above, to understand the impact of weather in the study, the parameters of minimum and maximum daily temperature, daily humidity and daily precipitation were monitored as controls. All measured parameters were very similar between 2021 and 2022. This confirmed the validity of the study, demonstrating that the lower scores observed in the efficacy study year 2022 were not caused by differences in weather and consecutively altered insect-biting pressure, but by the horses' different allergic reactions to a comparable insect-biting pressure as in 2021.

TABLE 2
Average AUC for 2021 and 2022 and percentage of change

| Timeframe | Average 2021 AUC | Average 2022 AUC | % Change |
|---|---|---|---|
| Overall | 130.34 | 65.63 | −50% |
| (weeks 21-46) | | | |

### CONCLUSION

In conclusion, these results from severe real patients show that IV injection with ePB-MSCs is a safe and effective to a major extent treatment for horses suffering from insect bite hypersensitivity.

The present invention is in no way limited to the embodiments described in the examples and/or shown in the figures. On the contrary, methods according to the present invention may be realized in many different ways without departing from the scope of the invention.

1. A method for treatment of insect-bite hypersensitivity (IBH) in equines, said method comprising administering to an equine in need thereof mesenchymal stem cells (MSCs) or a pharmaceutical composition comprising an effective amount of MSCs for use in the treatment of IBH in equines.

2. The method according to claim 1, wherein said treatment is prophylactic, metaphylactic and/or therapeutic.

3. The method according to claim 1, wherein said IBH is caused by Culicoides spp.

4. The method claim 1, wherein said MSCs are native.

5. The method according to claim 1, wherein said MSCs are derived from blood.

6. The method according to claim 1, wherein said MSCs are autologous or allogeneic MSCs.

7. The method according to claim 1, wherein said MSCs are equine-derived.

8. The method according to claim 1, wherein said MSCs are intravenously administered.

9. The method according to claim 1, wherein a dose of 10⁵-10⁸ MSCs per equine is administered.

10. The method according to claim 1, wherein a single dose is administered.

11. The method according to claim 1, wherein multiple doses are administered with each dose being administered at different time points.

12. The method according to claim 1, wherein said treatment is treatment of one or more IBH-associated clinical symptoms in equines diagnosed with or suffering from IBH.

13. The method according to claim 12, wherein said one or more IBH-associated clinical symptoms are evaluated using an allergy scoring system.

14. The method claim 12, wherein the treatment results in an improvement in the one or more IBH-associated clinical symptoms comprising: pruritus, alopecia, dermatitis (acute or chronic lesions), weight loss or combinations thereof.

15. The method according to claim 12, wherein the treatment results in a decreased pruritus and/or decreased alopecia and/or decreased dermatitis in comparison to a non-treated or placebo treated control group of the same species.

16. The method according to claim 1,
wherein:
said MSCs measure negative for MHC class II molecules and/or CD45, and/or
said MSCs measure positive for mesenchymal markers CD29, CD44 and CD90 and measure negative for MHC class II molecules and CD45, and/or
said MSCs secrete immunomodulatory prostaglandin E2 (PGE₂) cytokine when present in an inflammatory environment or condition, and/or
said MSCs have an increased secretion of at least one of the molecules chosen of IL-6, IL-10, TGF-β, NO, or a combination thereof, and/or
said MSCs have a decreased secretion of IL-1 when present in an inflammatory environment or condition and compared to a cell having the same characteristics but not being subjected to said inflammatory environment or condition, and/or
said MSCs stimulate the expression of PgE₂, IL-6, IL-10, NO, or a combination thereof when in the presence of PBMCs and/or suppress the secretion of TNF-α, IFN-γ, IL-1, TGF-β, IL-13 or a combination thereof when in the presence of PBMCs.
